(19) United States
(12) Patent Application Publication
(10) Pub. No.: US 2006/0222709 A1
(43) Pub. Date: Oct. 5, 2006

(54) Metformin methods and formulations for treating chronic constipation

(75) Inventor: John Devane, Co. Roscommon (IE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: AGI Therapeutics Research Ltd.

(21) Appl. No.: 11/375,399

(22) Filed: Mar. 15, 2006

Related U.S. Application Data
(60) Provisional application No. 60/662,920, filed on Mar. 18, 2005.

(51) Int. Cl. A61K 9/24; A61K 31/155

(52) U.S. Cl. 424/472; 514/635

(57) ABSTRACT

The present invention is directed to methods and formulations for treating chronic constipation. The methods and formulations include, but are not limited to, methods and formulations for delivering effective concentrations of metformin for treating chronic constipation and further comprise at least one pharmaceutically acceptable ingredient to control the release of the metformin, wherein following administration, the release of metformin is distal to the gastrointestinal sites to achieve systemic absorption of metformin. The invention is also directed to treating constipation as a symptom associated with other diseases and conditions such as irritable bowel syndrome.

This application claims priority to U.S. Provisional Patent Application No. 60/662,920, filed Mar. 18, 2005.

The present invention is directed to methods and formulations for treating chronic constipation. The methods and formulations include, but are not limited to, methods and formulations for delivering effective concentrations of metformin for treatment. The methods and formulations further comprise at least one pharmaceutically acceptable ingredient to control the release of the metformin, wherein following administration, the release of metformin is distal to at least one site of metformin uptake in the gastrointestinal tract. The present invention also relates to treating constipation as a symptom associated with other diseases and conditions such as irritable bowel syndrome (IBS).

Constipation occurs in up to 30% of the population. This symptom accounts for 1.2% of physician visits in the United States and is most frequently treated by primary care physicians. It is more common in females and increases with age. D. A. Drossman, The Functional Gastrointestinal Disorders and the Rome III Process, 45 Gut II1-II5 (Suppl. II 1999). There is also evidence to suggest that non-whites and persons of lower socioeconomic status are more likely to report chronic constipation. Almost a third of children with severe constipation will continue to suffer with symptoms beyond puberty.

Constipation comprises a group of functional disorders, which present as persistent, difficult, infrequent or seemingly incomplete defecation. Constipation has commonly been defined by three methods: 1) symptoms, in descending order of frequency, straining, hard stools, or scybala, unproductive calls (“want to but can't”), infrequent stools, incomplete evacuation; 2) parameters of defecation outside the 95^{th} percentile, e.g., less than three bowel movements per week, daily stool weight less than 35 g/day, or straining greater than 25% of the time; or 3) physiological measures such as prolonged whole gut transit or colonic transit as determined for instance by radio-opaque markers. D. A. Drossman, The Functional Gastrointestinal Disorders and the Rome III Process, 45 Gut II1-II5 (Suppl. II 1999).

As provided in Brooks Cash & William D. Chey, Update on the Management of Chronic Constipation: What Differentiates Chronic Constipation From IBS With Constipation, Medscape, at http://www.medscape.com/viewprogram/3375_pnt (Aug. 26, 2004), a variety of conditions and medications can be associated with chronic constipation, for example, primary or idiopathic constipation can be broadly divided into slow-transit constipation (i.e., colonic inertia) and dyssynergic defecation (i.e., anismus, outlet obstruction, pelvic floor dysfunction, pelvic floor dyssynergia, defecatory dysfunction). Physiologic abnormalities in patients with slow-transit constipation can include abnormal postprandial colonic motor function, autonomic dysfunction, and reduced numbers of colonic enterochromaffin cells and interstitial cells of Cajal. Dyssynergic defecation can occur as a consequence of the inability to coordinate actions of the abdominal musculature, anorectum, and pelvic floor musculature. An example is puborectalis dyssynergia, wherein the puborectalis sling fails to relax or paradoxically contracts with straining. This prevents straightening of the anorectal angle, which should precede the normal passage of stool. Structural abnormalities, such as a large rectocele, rectal intussusception, and obstructing sigmoidocele, can also contribute to constipation.

In addition, there can be significant overlap between patients with chronic constipation and irritable bowel syndrome-constipation (IBS-C) or constipation-dominant IBS. IBS can be characterized by abdominal discomfort or pain, bloating, and disturbed defecation. This disturbed defecation can take the form of constipation (IBS-C), diarrhea (IBS-D), or mixed/alternating bowel habits (IBS-M) with roughly equivalent distribution of the three subtypes.

Chronic constipation can also be a result of medications, endocrine disorders, and neurological disorders. For example, medications such as opiates, psychotropics, anticonvulsants, anticholinergics, dopaminergics, calcium channel blockers, bile acid binders, nonsterodial anti-inflammatory drugs, and supplements, i.e., calcium and iron, can initiate the onset of chronic constipation. Endocrine disorders such as diabetes mellitus, hypothyroidism, hyperparathyroidism, and pheochromocytoma similarly provoke the onset of chronic constipation. Moreover, chronic constipation can occur with both systemic (e.g., diabetic neuropathy, Parkinson's disease and Shy-Drager syndrome) and traumatic (e.g., spinal chord lesions) neurological disorders and. The term “constipation” as used herein, thus, encompasses conditions commonly identified as chronic constipation, functional constipation, chronic functional constipation, constipation, IBS-C, and/or other (non-chronic) constipation states.

Therapies for Chronic Constipation

The medical management of chronic constipation comprises lifestyle modifications in, e.g., diet and exercise, the use of bulking agents, e.g., psyllium, bran, methylcellulose, and calcium polycarbophil, and the administration of laxatives, including osmotic (e.g., polyethyleneglycol (PEG), lactulose, sorbitol, magnesium and phosphate salts), stimulants (e.g., senna-based and bisacodyl-based), and 5-hydroxytryptamine 4 (serotonin, 5-HT₄) receptor agonists (e.g., tegaserod).

Bulking Agents

Dietary fiber supplementation is believed to benefit constipated subjects by improving gastrointestinal transit and producing larger, softer stools. Dietary fiber supplementation can be, for example, achieved by increasing the ingestion of fiber-rich foods or by providing commercially available fiber supplements. Patients with chronic constipation can require greater doses of fiber than healthy volunteers to produce similar increases in stool volume and transit. Patients with severe colonic inertia or documented dyssynergic defecation can be less likely to improve with fiber.

Bulking agents can include psyllium, wheat bran, calcium polycarbophil, and methylcellulose. Three placebo-controlled trials of psyllium in patients with chronic constipation demonstrated improvements in stool frequency and consistency at doses ranging from 10 g/day to 24 g/day. L. J. Cheskin et al., Mechanisms of Constipation in Older Persons and Effects of Fiber Compared with Placebo, 43 J. American Geriatric Society 666-69 (1995); G. C. Fenn et al., A General Practice Study of the Efficacy of Regulanin Functional Constipation, 40 British J. Clinical Practice 192-97 (1986); and W. Ashraf et al., Effects of Psyllium Therapy on Stool Characteristics, Colon Transit and Anorectoal Function in Chronic Idiopathic Constipation, 9 Aliment Pharmacology & Therapeutics 639-47 (1995).

Despite the popularity of bran as a treatment for constipation, no randomized trials have shown improvements in stool frequency or consistency in patients with chronic constipation. There are no placebo-controlled trials examining calcium polycarbophil or methylcellulose in chronic constipated patients. In small trials comparing these agents versus psyllium, the data fail to demonstrate differences between agents in changes in stool frequency or consistency. R. Mamtani et al., A Calcium Salt of an Insoluble Synthetic Bulking Laxative in Elderlty Bedridden Nursing Home Residents, 8 J. American College Nutrition 554-56 (1989); and J. W. Hamilton et al., Clinical Evaluation of Methylcellulose as a Bulk Laxative, 33 Dig. Dis. Sci. 993-98 (1988).

Issues pertaining to convenience, palatability, and dose-dependent side effects (e.g., distention, bloating, and flatulence) limit patient compliance with instructions to use fiber supplements. Rare cases of anaphylaxis have been reported in patients taking psyllium.

Stool Softeners and Laxatives

Stool softeners can include, for example, dioctyl sodium sulfosuccinate and dioctyl calcium sulfosuccinate. Although these agents are commonly recommended for patients with constipation, there is little evidence to support their efficacy. Of four randomized controlled trials that evaluated stool softeners in patients with chronic constipation, only one, of three weeks' duration, found improvements in stool frequency compared with placebo. A. M. Fain et al., Treatment of Constipation in Geriatric and Chronically III Patients: A Comparison, 71 South Med. J. 677-80 (1978). In another trial, psyllium was found to be superior to dioctyl sodium sulfosuccinate in improving stool frequency. J. W. McRorie et al., Psyllium is Superior to Docusate Sodium for Treatment of Chronic Constipation, 12 Aliment Pharmacology & Therapeutic 491-97 (1998).

Laxatives can be broadly divided into two categories: osmotic and stimulant laxatives. Examples of oral osmotic laxatives include poorly absorbed saccharides and saccharide derivatives, such as lactulose and sorbitol. These agents can increase stool volume and water content and, in so doing, stimulate peristalsis. Two trials have demonstrated that lactulose can be more effective than placebo at improving stool frequency and consistency. J. F. Sannders, Lactulose Syrup Assessed in a Double-Blind Study of Elderly Constipated Patients, 26 J. American Geriatric Society 236-39 (1978); A. Wesselius-De Casparis et al., Treatment of Chronic Constipation with Lactulose Syrup: Results of a Double-Blind Study, 9 Gut 84-86 (1968). Unfortunately, osmotic laxatives can sometimes be associated with the development of abdominal cramping and bloating.

Other examples of osmotic laxatives include incompletely absorbed salts comprising magnesium or sodium phosphate that produce a laxative effect by inducing a net flux of water into the bowel. Surprisingly, there are no randomized placebo-controlled trials assessing the efficacy of these agents in patients with chronic constipation. Hypermagnesemia and hyperphosphatemia can occur with these agents, such as in persons with renal disease or in the elderly.

Yet another example of an osmotic laxative is polyethylene glycol (PEG), which recently became available for the treatment of patients with occasional constipation. A number of randomized placebo-controlled trials in patients with constipation demonstrated significant improvements in stool frequency and consistency with PEG at doses of ranging from 17 g/day to 35 g/day. R. I. Andorsky and F. Goldner, Colonic Lavage Solution (Polyethylene Glycol Electrolyte Lavage Solution) as a Treatment for Chronic Constipation: A Double-Blind, Placebo-Controlled Study, 85 American J. Gastroenterol. 261-65 (1990); M. V. Cleveland et al., New Polyethylene Glycol Laxative for Treatment of Constipation in Adults: A Randomized, Double-Blind, Placebo-Controlled Study, 94 South Med. J. 478-81 (2001); E. Corazziari et al., Small Volume Isomotic Polyethylene Glycol Electrolyte Balanced Solution (PMF-100) in Treatment of Chronic Nonorganic Constipation, 41 Dig. Dis. Sci. 1636-42 (1996); and E. Corazziari et al., Long Term Efficacy, Safety, and Tolerability of Low Daily Doses of Isosmotic Polyethylene Glycol Electrolyte Balanced Solution (PMF-100) in the Treatment of Functional Chronic Constipation, 46 Gut 522-26 (2000). PEG, however, is not currently approved for use in treating chronic constipation.

Laxatives in the second category, stimulant laxatives, usually comprise bisacodyl, sodium picosulfate, or anthraquinone derivatives, such as cascara sagrada and senna. These agents have effects on bowel secretion and motility. There are no randomized placebo-controlled trials that assess the efficacy of stimulant laxatives in patients with chronic constipation. One comparative trial suggested that an “irritant laxative” was not as effective as lactulose in patients with constipation. P. Connolly et al., Comparison of “Duphalac” and “Irritant” Laxatives During and After Treatment of Chronic Constipation: A Preliminary Study, 2 Current Medical Research Opinions 620-25 (1974). Anthraquinone laxatives can induce melanosis coli, a reversible process that occurs as a consequence of colonic epithelial cell apoptosis and deposition of lipofuscin in macrophages.

Additional Treatments

Tegaserod, 3-(5-methoxy-1H-indol-3-ylmethylene)-N-pentylcarbazimidamide hydrogen maleate, is a 5-HT₄ (serotonin) agonist that stimulates the peristaltic reflex as well as chloride secretion and can affect visceral sensation. A number of, randomized, placebo-controlled trials indicate that tegaserod at a dose of 6 mg twice daily effectively improves global and individual symptoms in women patients with IBS-C. W. D. Chevy, Tegraserod and Other Sterotonergic Agents: What is the Evidence?, 3 Review Gastroenterol Disorders S35-S40 (2003); S. A. Muller-Lissner et al., Tegaserod, a 5-HT4 Receptor Partial Agonist, Relieves Symptoms of Irritable Bowel Syndrome in Patients with Abdominal Pain, Bloating and Constipation, 15 Aliment Pharmacology & Therapeutics 1655-66 (2001). Similar benefits, however, have not been demonstrated in male IBS patients.

In August 2004, the U.S. Food and Drug Administration approved a supplemental indication for tegaserod, allowing its use in the treatment of chronic idiopathic constipation in patients younger than 65 years. Tegaserod, however, must be used with caution including a specific precaution in relation to ischemic colitis.

In view of the foregoing, there remains a need in the art for pharmaceutical methods and formulations that can provide an effective, well tolerated treatment of chronic constipation that avoids at least one of the many side effects and limitations associated with current therapies. The present disclosure solves at least one of the problems in the prior art and provides such methods and formulations for the treatment of chronic constipation.

The present invention is directed to metformin hydrochloride, i.e., dimethylbiguanide. Metformin is a biguanide that is conventionally used as an oral antihyperglycaemic agent in the management of non-insulin dependent diabetes (NIDDM). C. J. Dunn and D. H. Peters, Metformin: A Review of Its Pharmacological Properties and Therapeutic Use in Non-Insulin-Dependent Diabetes Mellitus, 49 Drugs 721-49 (1998). As used herein, the term “metformin” means metformin and any pharmaceutically acceptable salt thereof, e.g., metformin hydrochloride.

Metformin can reduce blood glucose levels, predominantly by improving hepatic and peripheral tissue sensitivity to insulin without affecting the secretion of that hormone. Metformin is the only member of the biguanide class currently approved. Other members of the biguanide class, e.g., phenformin and buformin, are no longer available for clinical use due, in part, to the unacceptably high incidence of lactic acidosis.

Lactic acidosis is an accumulation of lactate caused by damage to mitochondria. Like other biguanides, metformin might also have side effect, which can be more likely to occur in subjects with elevated levels of metformin and/or in subjects with compromised cardiac, pulmonary, hepatic and/or renal function.

Metformin can be used to treat NIDDM both as a monotherapy or as a combination therapy with other treatments of diabetes such as with certain sulfonylureas, e.g., glipizide and glyburide, and with thiazolidinediones, e.g., rosigliazone.

After oral administration, metformin can have an absolute bioavailability, i.e., the availability of metformin in systemic circulation after non-intravenous administration, ranging from 50% to 60%. Renal clearance is approximately 3.5 times greater than creatinine clearance, which indicates that tubular secretion is the major route of metformin elimination. 2003 Physicians' Desk Reference 1079. The plasma elimination half-life ranges from 4.0 to 8.7 hours, but can be prolonged in patients with renal impairment and correlates with creatinine clearance. Id.

Administration of conventional formulations of metformin can be initiated at a doses ranging from 0.5 g/day to 1 g/day in divided doses with or after meals. The dose can be gradually increased as necessary, for example, to a maximum of 2.5 g/day (5×500 mg tablets) or 2.55 g/day (3×850 mg tablets), although daily doses up to 3 g/day are used in countries other than the United States. Id.

Acute, reversible, adverse effects, mainly of gastrointestinal tract (GI) origin, occur in up to 20% of patients. For example, patients treated with metformin have a markedly higher prevalence diarrhea (20%) than those treated with other oral hypoglycemic agents (6%). P. Bytzer et al., Oral Hypoglycaemic Drugs and Gastrointestinal Symptoms in Diabetes Mellitus, 15 Aliment Pharmacology & Therapeutics 137-142 (2001); see also, P. Dandonna et al., Diarrhea and Metformin in a Diabetic Clinic, 6 Diabetes Care 472-474 (1983). Efforts to minimize GI side effects include administering metformin with food and reducing the dose. C. J. Dunn and D. H. Peters, Metformin: A review of Its Pharmacological Properties and Therapeutic Use in Non-Insulin-Dependent Diabetes Mellitus, 49 Drugs 721-749 (1998).

The mechanism through which metformin causes these GI effects is, however, not fully understood. According to researchers, metformin can effect GI motility or impair absorption. P. Dandonna et al., Diarrhea and Metformin in a Diabetic Clinic, 6 Diabetes Care 472-474 (1983). Because metformin chemically resembles 5-HT₃ receptor agonists, researchers suggest the drug's GI side effects can be due to activation of 5-HT₃ receptors. Based on a recent study, however, this was not the case. Irene S. Hoffmann et al., Ondansetron and Metformin-Induced Gastrointestinal Side Effects, 10 American J Ther. 447-451 (2003).

Extended release forms of metformin have been developed for once daily dosing. For example, GLUCOPHAGE® XR (500 mg and 750 mg tablets) and generic forms are marketed in the United States, as indicated in the Orange Book, and worldwide. U.S. Pat. Nos. 6,475,521 and 6,660,300 are directed to the biphasic release of the antidiabetic metformin HCl salt, i.e., GLUCOPHAGE® XR.

One benefit associated with extended release forms is a reduced incidence of GI side effects such as diarrhea. For example, the incidence of diarrhea in clinical trials of immediate-release metformin (GLUCOPHAGE®) in the approved label was 53.2% versus 11.7% for placebo with 6% discontinuation. 2003 Physicians' Desk Reference 1079-85. In contrast, the incidence in clinical trials of the extended-release label was 9.6% versus 2.6% for placebo. See id. In a retrospective patient chart review report, patients switched from immediate-release metformin to metformin XR experienced fewer GI side effects on comparable doses of the extended-release metformin. Lawrence Blonde et al., Gastrointestinal Tolerability of Extended-Release Metformin Tablets Compared to Immediate-Release Metformin Tablets: Results of a Retrospective Cohort Study, 20 Current Medical Research and Opinions 565-572 (2004).

Metformin is protonated at physiologic pH and when ionized tends to be adsorbed by the intestinal epithelium. Moreover, oral bioavailability of metformin ranges from 40% to 60%, decreasing with increasing dosage, i.e., there is not a direct relationship between plasma drug concentration and magnitude of effect. This suggests a saturable absorption process and/or absorption limited by permeability/transit time. Despite these unique pharmacokinetic and pharmacodynamic properties, the bioavailability of extended release forms of metformin was not significantly reduced compared to immediate release forms. David Stepensky et al., Preclinical Evaluation of Pharmacokinetic-Pharmacodynamic Rationale for Oral CR Metformin Formulation, 71 J Controlled Release 107-115 (2001). Thus, GLUCOPHAGE® XR, while demonstrating a delayed maximum plasma concentration (Tₘₐₓ) of 7 hours and a lower mean maximum plasma concentration (Cₘₐₓ), was equivalent in extent of exposure, i.e., area under the plasma concentration-time curve (AUC). 2003 Physicians' Desk Reference 1080-81. In another study, three different extended-release forms of metformin were compared with GLUCOPHAGE® and showed only small reductions in AUC (7.91 for GLUCOPHAGE ® versus 6.24, 7.07, and 6.43 for the extended release forms). P. J. Pentikainen, Bioavailability of Metformin, Comparison of Solution, Rapidly Dissolving Tablet, and Three Sustained Release Products, 24 Int'l J Clinical Pharmacology Ther. Toxicology 213-220 (1986).

U.S. Patent Application Publication No. 2004/0161461 describes another example of controlled release metformin formulations used to treat NMIDD. This formulation is directed to an extended-release metformin tablet with a high drug loading in the core and a rate-controlling coating that is insoluble but permeable resulting in a classic controlled-release profile with up to 60% drug release in two hours. Further, for example, U.S. Pat. No. 6,495,162 describes an osmotic controlled-release metformin tablet comprising a semipermeable-membrane coating with at least one passageway in the membrane and an absorption enhancer. This tablet formulation was designed to be administered with meals without reducing systemic exposure (i.e., bioavailability) when taken with food.

Still other metformin formulations are described in U.S. Patent Application Publication No. 2004/0096499, which describes a fixed combination dosage form of metformin and various low dose antidiabetic agents including, for example, acarbose, and U.S. Pat. No. 6,451,808, which describes a combination formulation of metformin with a 5-HT₃ antagonist to suppress the GI side effects including, for example, emesis.

Metformin has also been suggested in various other methods and formulations that extend the systemic exposure and/or reduce possible GI side effects such as U.S. Pat. No. 6,790,459, which describes a method for treating diabetes with a once-daily controlled-release form of metformin resulting in a bioavailability (AUC) equivalent to immediate-release forms; International Patent Application WO 00/28989, which describes a composition for treating diabetes comprising a modified-release thiazolidinedione insulin sensitizer and another antidiabetic agent (metformin); U.S. Patent Application Publication No. 2004/00175424, which describes a microcapsules dosage form of metformin that prolongs the release but excludes delayed-release dosage forms; U.S. Patent Application Publication No. 2004/0022849, which describes an oral dosage form of metformin based on two controlled-release mechanisms acting in series; and U.S. Pat. No. 6,022,562, which describes a dosage form comprising microcapsules of small size that are retained in the small intestine thereby achieving substantial and prolonged absorption and systemic exposure of the drug. All known metformin formulations provide for extended systemic exposure and prolonged absorption, i.e., they are retained in the GI tract (gastroretentive) to increase systemic absorption.

Gastroretentive formulations are known in the art, for example U.S. Pat. Nos. 5,007,780, 5,972,389, and 5,582,837, which describe a plurality of solid particles dispersed in a hydrophobic, water-swellable polymer that promotes gastric retention. U.S. Pat. Nos. 5,651,985 and 6,306,439 describe methods for making gastroretentive dosage forms by intensive mixing of polymers comprising lactam groups and polymers comprising carboxyl groups thereby conferring marked and unexpected swelling properties on the dosage form. U.S. Pat. No. 6,261,601 describes a dosage form that incorporates a gas-generating component that contributes to the expansion of the matrix. And U.S. Pat. No. 6,685,962 describes a gastroretentive dosage form that incorporates both degradable polymers and non-degradable polymers in a drug matrix that is attached to a membrane so that the combined unit is not evacuated from the stomach for extended periods of time.

Additional references disclose the incorporation of metformin into various gastroretentive dosage forms prolonging the release in, for example, the stomach and upper small intestine, such as U.S. Pat. Nos. 6,723,340, 6,682,759, 6,635,280, 6,488,962, and U.S. Patent Application Publication No. 2003/0104062.

In accordance with the present disclosure, novel methods and formulations are provided for treating chronic constipation and/or constipation as a symptom associated with other diseases and/or conditions such as IBS. The formulations of the invention release metformin outside the site of absorption employed for treating diabetes, e.g., bypassing absorption in the stomach and, further, for example, bypassing absorption in the upper small intestine. These and other embodiments of the present invention are achieved by methods and formulations treating chronic constipation in a subject in need of such treatment, comprising administering to a subject a dosage formulation comprising an effective amount of metformin, or a pharmaceutically acceptable salt thereof, and at least one pharmaceutically acceptable ingredient to control the release of the metformin, wherein following administration, the dosage formulation releases the metformin distal to the gastrointestinal sites for systemic absorption.

Chronic constipation can be caused by conditions including, but not limited to, lifestyle habits, e.g., low dietary fiber and immobility, diseases of the peripheral and central nervous system, anatomic gastrointestinal obstructive lesions, endocrine disorders, metabolic disturbances, myotonic dystrophy, use of certain drugs, and/or can be a symptom of any of the foregoing conditions. Chronic constipation can be treated with the administration of a modified-release formulation of metformin, or a pharmaceutically acceptable salt thereof.

In at least one embodiment, the present invention is also directed to methods for treating chronic constipation and/or treating constipation as a symptom associated with another disease and/or condition, for example, IBS, in a subject in need of such treatment. These methods include administering to the subject a dosage formulation comprising an effective amount of metformin, or a pharmaceutically acceptable salt thereof, and at least one pharmaceutically acceptable ingredient to control the release of the metformin, wherein following administration, the dosage formulation releases the metformin distal to gastrointestinal sites for systemic absorption such as bypassing the release of metformin in the stomach.

In at least one embodiment of the present invention, the metformin is present in a pharmaceutical dosage formulation that can comprise at least one pharmaceutically acceptable ingredient for controlled release. The controlled release enables the metformin to be released distal to the gastrointestinal (GI) tract site for systemic absorption of the drug, e.g., bypassing the stomach.

In all embodiments, the metformin can comprise substantially pure metformin, or a pharmaceutically acceptable salt thereof. The metformin, or pharmaceutically acceptable salt thereof, can be administered in combination with at least one additional pharmaceutically active compound. In some embodiments, the at least one additional pharmaceutically active compound is capable of relieving constipation.

It is to be understood that both the foregoing general description and the following detailed description are exemplary and explanatory and are not restrictive of the present invention, as claimed.

The present invention is directed to formulations comprising metformin and methods of their use. Although not wishing to be bound to any particular theory, it is believed that the presence of a therapeutically effective amount of metformin, or a pharmaceutically acceptable salt thereof, reduces the incidence of chronic constipation and/or the symptom of constipation associated with other diseases and/or conditions. It is believed that when metformin is administered in a modified-release dosage form, metformin reduces the incidence of chronic constipation. In accordance with the methods and formulations of the present disclosure, it has been determined that subjects suffering from chronic constipation and/or exhibiting constipation as a symptom of diseases and/or conditions are able to reduce the incidence of constipation with the administration of a modified-and/or delayed-release formulation of metformin such that the release of metformin occurs outside the site of absorption employed for treating diabetes, e.g., bypassing the stomach and, further for example, bypassing the upper small intestine.

In order to further describe the present invention, the following terms and definitions are provided.

As used herein, the phrase “modified-release” formulation or dosage form includes a pharmaceutical preparation that achieves a desired release of the drug from the formulation other than immediate release. For example, a modified-release formulation can restrict release and/or control the site of release of a therapeutically effective dose of an active compound in a subject. In addition, a modified-release formulation can also be designed to delay the release of the active compound for a specified period. Such compounds are referred to herein as “delayed-release” compounds. Modified-release formulations can exhibit properties of delayed formulations.

As used herein, the phrase “sustained-release” formulation or dosage form means a formulation capable of gradual release of an active agent, i.e., drug, over a period of time, allowing for a sustained effect.

As used herein, the term “metformin” includes metformin, and any pharmaceutically acceptable salt thereof.

As used herein, the phrase “pharmaceutically acceptable ingredient” includes components that are compatible with the other ingredients in a pharmaceutical formulation, for example, the active ingredients, and not injurious to the subject when administered in acceptable amounts. Pharmaceutically acceptable ingredients that can be mentioned include, but are not limited to, for example, carriers, fillers, extenders, binders, disintegrating agents, solution-retarding agents, absorption accelerators, wetting agents, absorbents, lubricants, stabilizers, coloring agents, buffering agents, dispersing agents, preservatives, organic acids, water-soluble and water-insoluble polymers, enteric and non-enteric agents, and coatings.

As used herein, the phrase “pharmaceutically acceptable salt” includes salts that are physiologically tolerated by a patient. Such salts can be prepared from inorganic salts or bases and/or organic acids or bases. Examples of these acids and bases are well known to those of ordinary skill in the art.

As used herein, the phrase “therapeutically effective amount” means the amount of metformin (or a pharmaceutically acceptable salt thereof)that alone and/or in combination with additional drugs provides a benefit in the prevention, treatment, and/or management of chronic constipation and/or a constipation-related symptom.

The present invention further includes methods of preventing, treating, and/or managing chronic constipation and/or constipation-related symptoms associated with other diseases and/or conditions. In some embodiments, the administration of metformin or a pharmaceutically acceptable salt thereof reduces the risk of systemic effects, for example, at least one side effect, e.g., lactic acidosis, associated with the administration of a conventional formulation of metformin, i.e., a formulation for treating diabetes (non-insulin dependent diabetes. One skilled in the art will appreciate that, when metformin is administered to treat constipation, systemic absorption can result in hypoglycemia, which would be an undesirable side effect.

In certain embodiments, the metformin methods and formulations of the invention can be administered using a dosage formulation chosen from modified-release forms, for example, a formulation that controls the site of release. These formulations release the metformin distal to gastrointestinal sites at which metformin is usually systemically absorbed, for example, bypassing the release of metformin in the stomach, i.e., the site of primary absorption when metformin is administered to treat diabetes. For example, in some embodiments, the modified-release dosage form is designed to bypass release in the stomach and/or the upper part of the GI tract, i.e., the formulation is not gastroretentive and/or upper small intestine-retentive. In various embodiments, the invention provides methods and formulations that do not release metformin, or a pharmaceutically acceptable salt thereof, in an area defined by the stomach and the upper small intestine.

In some embodiments, the dosage formulation of metformin can be administered to a subject in need of such treatment. In certain embodiments, the metformin formulation can be administered to the subject in a fasting state.

The dosage formulations of the invention can be suitable for treating and/or preventing conditions or diseases that exhibit constipation as a symptom. Such diseases and/or conditions include, but are not limited to, IBS and those that are typically treated and/or prevented with conventional bulking agents and laxatives.

Among the routes of administration for dosage formulations according to the invention are, for example, oral administration and any other route that can be used to administer a modified and/or delayed release formulation designed to release the drug to sites in the GI tract distal to the sites of systemic drug absorption.

For oral administration, the metformin can be formulated into a liquid dosage form. Suitable formulations include emulsions, microemulsions, solutions, suspensions, syrups, and exlixirs. These formulations optionally include diluents commonly used in the art, such as, for example, water or solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcholol, polyethylene glycols, fatty acid esters of sorbitan, and mixtures thereof. In addition, the liquid formulations optionally include adjuvants such as wetting, emulsifying, suspending, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations can be delivered as-is, or can be provided in hard or soft capsules.

Soft Gelatin Capsules

The formulations of the present invention can be prepared as liquids that can be filled into soft gelatin capsules. For example, the liquid can include a solution, suspension, emulsion, microemulsion, precipitate, or any other desired liquid media carrying the metformin. The liquid can be designed to improve the solubility of the metformin upon release, or can be designed to form a drug-comprising emulsion or a dispersed phase upon release. Examples of such techniques are well known in the art. Soft gelatin can be coated, as desired, with a functional coating to delay the release of the drug.

The compositions of the invention can also be formulated into other dosage forms that modify the release of the active agent. Examples of suitable modified-release formulations that can be used in accordance with the present invention include, but are not limited to, matrix systems, osmotic pumps, and membrane-controlled dosage forms. These formulations can comprise metformin or a pharmaceutically acceptable salt thereof. Suitable pharmaceutically acceptable salts are discussed above. Each of these types of dosage forms are briefly described below. A more detailed discussion of such forms can also be found in, for example, The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992), the relevant contents of each of which is hereby incorporated by reference for this purpose.

Matrix-Based Dosage Forms

In some embodiments, the modified-release and/or delayed-release formulations of the invention are provide as matrix-based dosage forms. Matrix formulations according to the invention can include hydrophilic, e.g., water-soluble, and/or hydrophobic, e.g., water-insoluble, polymers. The matrix formulations of the invention can be prepared with functional coatings, which can be enteric, e.g., exhibiting a pH-dependent solubility, or non-enteric, e.g., exhibiting a pH-independent solubility.

Matrix formulations of the invention can be prepared by using, for example, direct compression or wet granulations. For example, U.S. Pat. No. 6,495,162 describes a new compression manufacturing process for metformin tablets, which incorporates a sustained release polymer material that results in an initial burst of drug and a slower release. A functional coating, as noted above, can then be applied in accordance with the invention. Additionally, a barrier or sealant coat can be applied over a matrix tablet core before a functional coating is applied. The barrier or sealant coat can serve the purpose of separating an active ingredient from a functional coating, which can interact with the active ingredient, or it can prevent moisture from contacting the active ingredient. Details of barriers and sealants are provided below.

In a matrix-based dosage form in accordance with the present invention, the metformin and the at least one pharmaceutically acceptable ingredient are dispersed within a polymeric matrix, which typically comprises at least one water-soluble polymer and at least one water-insoluble polymer. The drug can be released from the dosage form by diffusion and/or erosion. Such matrix systems are described in detail by Wise and Kydonieus, supra.

Suitable water-soluble polymers include, but are not limited to, polyvinyl alcohol, polyvinylpyrrolidone, methylcellulose, hydroxypropylcellulose, hydroxypropylmethyl cellulose, or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride), or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethylcellulose, alginates, gelatin, polyvinylpyrrolidone, sucrose, and acacia; humectants, such as glycerol; disintegrating agents, such as agar, calcium carbonate, potato and tapioca starch, alginic acid, certain silicates, EXPLOTAB™, crospovidone, and sodium carbonate; solution-retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients can have more than one role or function, or be classified in more than one group; the classifications are descriptive only, and not intended to limit any use of an exemplified excipient.

In some embodiments, a matrix-based dosage form can comprise metformin; a filler, such as starch, lactose, or microcrystalline cellulose (AVICEL™); a binder/controlled-release polymer, such as hydroxypropyl methylcellulose or polyvinyl pyrrolidone; a lubricant, such as magnesium stearate or stearic acid; a surfactant, such as sodium lauryl sulfate or polysorbates; and a glidant, such as colloidal silicon dioxide (AEROSIL™) or talc. In certain embodiments, a disintegrant such as EXPLOTAB™, crospovidone, or starch is also included.

The amounts and types of polymers, and the ratio of water-soluble polymers to water-insoluble polymers, in the presently disclosed formulations are generally selected to achieve a desired release profile of metformin, as described below. For example, by increasing the amount of water-insoluble polymer relative to the amount of water-soluble polymer, the release of the drug can be delayed or slowed. This is due, in part, to an increased impermeability of the polymeric matrix, and, in some cases, to a decreased rate of erosion during transit through the GI tract.

Osmotic Pump Dosage Forms

In various embodiments, the modified-release formulations of the present disclosure are provided as osmotic pump dosage forms. In an osmotic pump dosage form, a core comprising the metformin and optionally at least one osmotic excipient can be encased by a selectively permeable membrane having at least one orifice. The selectively permeable membrane is generally permeable to water, but impermeable to the drug. When body fluids contact the system, water penetrates the selectively permeable membrane into the core containing the drug and optional osmotic excipients. The osmotic pressure increases within the dosage form, and the drug is released through the orifice(s) in an attempt to equalize the osmotic pressure across the selectively permeable membrane.

In more complex pumps, the dosage form can comprise at least two internal compartments in the core. The first compartment can comprise the drug and the second compartment can comprise at least one polymer, which swells on contact with aqueous fluid. After ingestion, this polymer swells into the drug-comprising compartment, diminishing the volume occupied by the drug, thereby enabling one to optimize the delivery of the drug from the device at a controlled rate over an modified period or base delivery on the pH of the particular environment.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention can be formed by compressing a tablet of an osmotically active drug, or an osmotically inactive drug in combination with an osmotically active agent, and then coating the tablet with a selectively permeable membrane that is permeable to an exterior aqueous-based fluid but impermeable to the drug and/or osmotic agent.

At least one delivery orifice can be drilled through the selectively permeable membrane wall. Alternatively, the at least one orifice in the wall can be formed by incorporating leachable, pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane. This enables one to optimize the delivery of the drug from the device at a modified rate over an extended period or base delivery on the pH of the particular environment.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

As discussed above, the osmotic pump dosage form can comprise a second compartment comprising a swellable polymer. Suitable swellable polymers typically interact with water and/or aqueous biological fluids, which causes them to swell or expand to an equilibrium state. Useful polymers exhibit the ability to swell in water and/or aqueous biological fluids, retaining a significant portion of the imbibed fluids within their polymeric structure, so as to increase the hydrostatic pressure within the dosage form. The polymers can swell or expand to a very high degree, for example, exhibiting a 2- to 50-fold volume increase. The polymers can be non-cross-linked or cross-linked. In some embodiments, the swellable polymers are hydrophilic polymers. Suitable polymers include, but are not limited to, poly(hydroxy alkyl methacrylate) having a molecular weight of from about 30,000 to about 5,000,000; kappa-carrageenan; polyvinylpyrrolidone having a molecular weight of from about 10,000 to about 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from about 200 to about 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene, or isobutylene; water-swellable polymers of N-vinyl lactams; and/or mixtures of any of the foregoing.

The term “orifice” as used herein includes means and methods suitable for releasing the drug from the dosage form. The expression includes an aperture or orifice that has been bored through the selectively permeable membrane by mechanical procedures. Alternatively, an orifice can be formed by incorporating an erodible element, such as a gelatin plug, in the selectively permeable membrane. In such cases, erosion of the erodible element forms pores in the selectively permeable membrane through which the drug can pass. Such “passageway” formulations are described, for example, in U.S. Pat. Nos. 3,845,770 and 3,916,899, the relevant disclosures of which are incorporated herein by reference for this purpose.

The osmotic pumps useful in accordance with the invention can be manufactured by techniques known in the art. For example, the drug and other ingredients can be milled together and pressed into a solid having the desired dimensions (e.g., corresponding to the first compartment). The swellable polymer can then formed, placed in contact with the drug, and both can be surrounded with the selectively permeable agent. If desired, the drug component and polymer component can be pressed together before applying the selectively permeable membrane. The selectively permeable membrane can be applied by any suitable method, for example by molding, spraying, or dipping.

Membrane-Controlled Dosage Forms

The modified-release formulations of the present invention can also be provided as membrane-controlled formulations. Membrane-controlled formulations of the invention can be made by preparing a rapid release core, which can be a monolithic (e.g., tablet) or multi-unit (e.g., pellet) type, and coating the core with a membrane. The membrane-controlled core can then be further coated with a functional coating. In between the membrane-controlled core and the functional coating, a barrier or sealant can be applied. The barrier or sealant can alternatively, or additionally, be provided between the rapid release core and the membrane coating. Details of membrane-controlled dosage forms are provided below.

In certain embodiments, the metformin can be provided in a multiparticulate membrane-controlled formulation. Metformin can be formed into an active core by applying the drug to a nonpareil seed having an average diameter in the range of about 0.4 to about 1.1 mm or about 0.85 to about 1.00 mm. The metformin can be applied with or without additional excipients onto the inert cores, and can be sprayed from solution or suspension using a fluidized-bed coater (e.g., Wurster coating) or pan coating system. Alternatively, the metformin can be applied as a powder onto the inert cores using a binder to bind the metformin onto the cores. Active cores can also be formed by extrusion of the core with suitable plasticizers (described below) and any other processing aids as necessary.

In various embodiments, the modified-release formulations of the present invention comprise at least one polymeric material, which is applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate, cellulose propionate, cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, poly (ethylene oxide), poly (ethylene terephthalate), poly (vinyl isobutyl ether), poly (vinyl acetate), poly (vinyl chloride), or polyurethane, and/or mixtures thereof.

EUDRAGIT® polymers (available from Rohm Pharma) are polymeric lacquer substances based on acrylates and/or methacrylates. A suitable polymer that is freely permeable to the active ingredient and water is EUDRAGIT® RL. A suitable polymer that is slightly permeable to the active ingredient and water is EUDRAGIT® RS. Other suitable polymers that are slightly permeable to the active ingredient and water, and exhibit a pH-dependent permeability, include, but are not limited to, EUDRAGIT® L, EUDRAGIT® S, and EUDRAGIT® E.

EUDRAGIT® RL and RS are acrylic resins comprising copolymers of acrylic and methacrylic acid esters with a low content of quaternary ammonium groups. The ammonium groups are present as salts and give rise to the permeability of the lacquer films. EUDRAGIT® RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT® L is an anionic polymer synthesized from methacrylic acid and methacrylic acid methyl ester. It is insoluble in acids and pure water. It becomes soluble in neutral to weakly alkaline conditions. The permeability of EUDRAGIT® L is pH dependent. Above pH 5.0, the polymer becomes increasingly permeable.

In various embodiments comprising a membrane-controlled dosage form, the polymeric material can comprise methacrylic acid co-polymers, ammonio methacrylate co-polymers, or mixtures thereof. Methacrylic acid co-polymers such as EUDRAGIT® S and EUDRAGIT® L (Rohm Pharma) are particularly suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT® S and EUDRAGIT® L can be used as single components in the polymer coating or in combination in any ratio. By using a combination of the polymers, the polymeric material can exhibit a solubility at a pH between the pHs at which EUDRAGIT® L and EUDRAGIT® S are separately soluble.

The membrane coating can comprise a polymeric material comprising a major proportion (i.e., greater than 50% of the total polymeric content) of at least one pharmaceutically acceptable water-soluble polymer, and optionally a minor proportion (i.e., less than 50% of the total polymeric content) of at least one pharmaceutically acceptable water insoluble polymer. Alternatively, the membrane coating can comprise a polymeric material comprising a major proportion (i.e., greater than 50% of the total polymeric content) of at least one pharmaceutically acceptable water insoluble polymer, and optionally a minor proportion (i.e., less than 50% of the total polymeric content) of at least one pharmaceutically acceptable water-soluble polymer.

Ammonio methacrylate co-polymers such as EUDRAGIT® RS and EUDRAGIT® RL (Rohm Pharma) are suitable for use in the modified-release formulations of the present disclosure. These polymers are insoluble in pure water, dilute acids, buffer solutions, or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCl) groups in the polymer. Those polymers having EA:MMA:TAMCl ratios of 1:2:0.2 (EUDRAGIT® RL) are more permeable than those with ratios of 1:2:0.1 (EUDRAGIT® RS). Polymers of EUDRAGIT® RL are insoluble polymers of high permeability. Polymers of EUDRAGIT® RS are insoluble films of low permeability.

The amino methacrylate co-polymers can be combined in any desired ratio, and the ratio can be modified to modify the rate of drug release. For example, a ratio of EUDRAGIT® RS: EUDRAGIT® RL of 90:10 can be used. Alternatively, the ratio of EUDRAGIT® RS: EUDRAGIT® RL can be about 100:0 to about 80:20, or about 100:0 to about 90:10, or any ratio in between. In such formulations, the less permeable polymer EUDRAGIT® RS would generally comprise the majority of the polymeric material.

The amino methacrylate co-polymers can be combined with the methacrylic acid co-polymers within the polymeric material in order to achieve the desired delay in the release of the drug. Ratios of ammonio methacrylate co-polymer (e.g., EUDRAGIT® RS) to methacrylic acid co-polymer in the range of about 99:1 to about 20:80 can be used. The two types of polymers can also be combined into the same polymeric material, or provided as separate coats that are applied to the core.

In addition to the EUDRAGIT® polymers described above, a number of other such copolymers can be used to control drug release. These include methacrylate ester co-polymers (e.g., EUDRAGIT® NE 30D). Further information on the EUDRAGIT® polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114.

In addition to the EUDRAGIT® polymers discussed above, other enteric, or pH-dependent, polymers can be used. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

The coating membrane can further comprise at least one soluble excipient to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose, and sucrose, sugar alcohols such as lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The soluble excipient(s) can be used in an amount of from about 1% to about 10% by weight, based on the total dry weight of the polymer.

In some embodiments, the polymeric material can comprise at least one water-insoluble polymer, which is also insoluble in gastrointestinal fluids, and at least one water-soluble pore-forming compound. For example, the water-insoluble polymer can comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compound(s) can be uniformly or randomly distributed throughout the water insoluble polymer. Typically, the pore-forming compound(s) comprises about 1 part to about 35 parts for each about 1 to about 10 parts of the water insoluble polymers.

When such dosage forms come in to contact with the dissolution media (e.g., intestinal fluids), the pore-forming compound(s) within the polymeric material dissolves to produce a porous structure through which the drug diffuses. Such formulations are described in more detail in U.S. Pat. No. 4,557,925, which relevant part is incorporated herein by reference for this purpose. The porous membrane can also be coated with an enteric coating, as described herein, to inhibit release in the stomach.

In certain embodiments, such pore-forming modified-release dosage forms comprise metformin; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The polymeric material can also include at least one auxiliary agent such as a fillers, plasticizer, and/or anti-foaming agent. Representative fillers include talc, fumed silica, glyceryl monostearate, magnesium stearate, calcium stearate, kaolin, colloidal silica, gypsum, micronized silica, and magnesium trisilicate. The quantity of filler used can range from about 2% to about 300% by weight, or from about 20% to about 100%, based on the total dry weight of the polymer. In various embodiments, talc is the filler.

The coating membranes and functional coatings can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material can range from about 10% to about 50%, for example, about 10%, 20%, 30%, 40%, or 50%, based on the weight of the dry polymer.

Anti-foaming agents can also be included. In some embodiments, the anti-foaming agent is simethicone. The amount of anti-foaming agent used can comprise from about 0% to about 0.5% of the final formulation.

The amount of polymerto be used in the membrane-controlled formulations can be adjusted to achieve the desired drug delivery properties, including the amount of drug to be delivered, the rate and location of drug delivery, the time delay of drug release, and the size of the multiparticulates in the formulation. The amount of polymer applied can provide an about 10% to about 100% weight gain to the cores. In certain embodiments, the weight gain from the polymeric material ranges from about 25% to about 70%.

A polymeric membrane can include components in addition to polymers, such as, for example, fillers, plasticizers, stabilizers, or other excipients and processing aids. One example of an additional component of the membrane is sodium hydrogen carbonate, which can act as a stabilizer.

The combination of all solid components of the polymeric material, including co-polymers, fillers, plasticizers, and optional excipients and processing aids, can provide an about 10% to about 450% weight gain to the cores. In various embodiments, the weight gain is about 30% to about 160%.

The polymeric material can be applied by any known method, for example, by spraying using a fluidized bed coater (e.g., Wurster coating) or a pan coating system. Coated cores can be dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature, which can be above the glass transition temperature of the relevant polymer.

A sealant or barrier can also be applied to the polymeric coating. Alternatively, or additionally, a sealant or barrier layer can be applied to the core prior to applying the polymeric material. A sealant or barrier layer is generally not intended to modify the release of metformin, but might, depending on how it is formulated. Suitable sealants or barriers are permeable or soluble agents such as hydroxypropyl methylcellu lose, hyd roxypropyl cellulose, hyd roxypropyl ethylcellu lose, polyvinyl pyrrolidone, and xanthan gum. An outer sealant/barrier, for example, can be used to improve moisture resistance of the entire formulation. A sealant/barrier between the core and the coating, for example, can be used to protect the core contents from an outer polymeric coating that can exhibit pH-dependent or pH-independent dissolution properties. Additionally, there can be instances in which both effects are desired, i.e., moisture resistance and core protection, in which a sealant/barrier can be applied between the core and the polymeric membrane coating, and then outside the polymeric membrane coating.

Other agents can be added to improve the processability of a sealant or barrier layer. Such agents include talc, colloidal silica, polyvinyl alcohol, titanium dioxide, micronized silica, fumed silica, glycerol monostearate, magnesium trisilicate, and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY® WHITE Y-1-7000® and OPADRY® OY/B/28920 WHITE®, each of which is available from Colorcon Limited, England.

The present invention also provides an oral dosage form comprising multiparticulate metformin as hereinabove defined, in the form of caplets, capsules, particles for suspension prior to dosing, sachets, or tablets. The dosage form can be of any shape suitable for oral administration of a drug, such as spheroidal, cube-shaped, oval, or ellipsoidal.

While various pharmaceutical dosage forms, rate controlling polymers and processes of manufacture can be employed, in certain embodiments of the invention, the metformin formulation can be a small length, i.e., less than 10 mm, tablet or a multiplicity of tablets each with an enteric coating chosen from EUDRAGIT® L and S grades. Such a tablet does not swell significantly on exposure to aqueous conditions and suppresses the release of the metformin in a pH environment less than 5.5 and pH greater than 6.5, and thus, will prevent the release within the absorption window, i.e., the stomach and the upper small intestine.

All of the embodiments described above, including but not limited to, matrix-based, osmotic pump-based, soft gelatin capsules, and/or membrane-controlled forms, which can further take the form of monolithic and/or multi-unit dosage forms, can have a functional coating. Such coatings can serve the purpose of delaying the release of the drug for a predetermined time. For example, such coatings can allow the dosage form to pass through the stomach without being dissolved by stomach acid or digestive juices. Thus, such coatings can dissolve or erode upon reaching a desired point in the gastrointestinal tract, such as sites distal to the site of metformin systemic absorption from the GI tract.

Such functional coatings can exhibit pH-dependent or pH-independent solubility profiles. Those with pH-independent profiles can erode or dissolve away after a predetermined period, and the period can be related to the thickness and composition of the coating. Those with pH-dependent profiles, on the other hand, can maintain their integrity while in the acid pH of the stomach, but can quickly erode or dissolve upon entering the more basic upper intestine.

Thus, a matrix-based osmotic pump-based, or membrane-controlled formulation can be further coated with a functional coating that delays the release of the drug. For example, a membrane-controlled formulation can be coated with an enteric coating that delays the exposure of the membrane-controlled formulation until the upper intestine is reached. Upon leaving the acidic stomach and entering the more basic intestine, the enteric coating dissolves. The membrane-controlled formulation then is exposed to gastrointestinal fluid, and then releases the metformin over an extended period, in accordance with the present disclosure. Examples of functional coatings such as these are well known to those in the art.

In some embodiments, the metformin formulations can initially delay release of the drug. Following delay, the formulation can rapidly release the drug.

Dosages

One of skill in the art will recognize that the dosage required to produce a therapeutic effect can vary depending on the individual being treated and the severity of the condition. For example, the age, body weight, and medical history of the individual patient can affect the therapeutic efficacy of the therapy. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response.

In some embodiments, the methods and formulations of the present invention exhibit a relative bioavailability of the modified-release and/or delayed-release formulations lower than that of an immediate release formulations such as less than 75% and, further for example, less than 50%, upon administration to a subject.

In general, the total daily dosage of metformin in formulations of the invention can range from about 50 mg to about 3 g, or any whole number or fractional amount in between. In addition, the daily dosage of metformin in formulations of the invention can be chosen from single and divided doses. For example, a single dose can be formulated to comprise about 50, 60, 70, 80, 90, 100, 110, 120, 125, 130, 140, 150, 160, 170, 200, 300, 400, 500, 600, 700, 800 or 900 mg, 1, 2, or 3 g of metformin. In one embodiment, a single dose comprises about 500 mg of metformin.

The oral formulations of the present invention can be characterized by their dissolution profiles. One skilled in the art is familiar with the techniques used to determine such dissolution profiles. The standard methodologies set forth in the U.S. Pharmacopoeia, which methodologies are incorporated herein by reference in relevant part, can be used. For example, the dissolution profile can be measured in either a U.S. Pharmacopoeia Type I Apparatus (baskets) or a U.S. Pharmacopoeia Type II Apparatus (paddles).

Release Profiles

Some embodiments of the invention are directed to methods and formulations that employ a formulation having a modified/delayed release profile.

For example, the present invention includes formulations, and methods of their use, that exhibit modified and/or delayed release profiles showing negligible release, i.e., less than 10%, for at least 2 hours in pH media less than or equal to pH 5 and, further for example, in pH media less than or equal to pH 6.5.

Optimization of the metformin release profile can permit one to delay release of the metformin in a manner such that release can occur in sites distal to the duodenum in the GI tract. Further for example, the release of metformin can occur in sites distal to the jejunum in the GI tract. Still yet another example, the release of metformin can occur in sites distal to the ileum in the GI tract.

Any pharmaceutical formulation described above can further comprise at least one additional pharmaceutically active agent other than metformin. Such compounds can be provided to treat the same condition being treated with metformin, or a different one. Such drugs include, but are not limited to, acarbose, bulking agents, laxatives, tegaserod, and mixtures thereof. Those of skill in the art are familiar with examples of techniques for incorporating additional active agents, i.e., ingredients, into the pharmaceutical formulations. For example, in certain embodiments, a combination formulation can comprise metformin, or a pharmaceutically acceptable salt thereof, at least one pharmaceutically acceptable ingredient, and at least one additional pharmaceutically active compound such as acarbose. In some embodiments, acarbose can be included as an immediate-release and/or as a modified-release formulation and for example, can be administered in a fixed combination with metformin.

Alternatively, such additional pharmaceutical active agents can be provided in a separate formulation and co-administered to a patient with a metformin composition. Such separate formulations can be administered before, after, or simultaneously with the administration of metformin.

Other than in the Examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties to be obtained by the invention. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the disclosure are approximations, the numerical values set forth in the specific examples are reported as precisely as is conventional in the art. Any numerical value, however, inherently contains certain errors necessarily resulting from the standard deviation found in their respective testing measurements.

The present invention is further illustrated by reference to the following examples. It will be apparent to those skilled in the art that many modifications, both to materials and methods, can be practiced without departing from the purpose and scope of the present disclosure.

### EXAMPLES

### Example 1

### Uncoated Instant Release Metformin Tablet Formulations

| | | Formulation | | |
|---|---|---|---|---|
| | | A | B | C |
| Ingredient | Function | mg/tab | mg/tab | mg/tab |
| Metformin | Active | 500.0 | 500.0 | 500.0 |
| Lactose | Diluent | 114.0 | 64.0 | 48.0 |
| Sodium Starch | Disintegrant | 80.0 | 70.0 | 66.0 |
| Glycolate | | | | |
| Avicel PH101 | Binder | 114.0 | 64.0 | 48.0 |
| | Diluent | | | |
| Colloidal Silicon | Glidant | 2.0 | 2.0 | 2.0 |
| Dioxide | | | | |
| Magnesium Stearate | Lubricant | 20.0 | 20.0 | 20.0 |
| Povidone (PVP, | Binder | 50.0 | 50.0 | 50.0 |
| polyvinylpyrollidone) | | | | |
| Isopropyl Alcohol | Solvent | N/A | N/A | N/A |
| (IPA)* | | | | |
| Total (mg) | N/A | 880.0 | 770.0 | 734.0 |

*Removed during processing.

Manufacturing Process

Weigh the ingredients using a suitable balance.

Place metformin, 50% of the Avicel, and 50% of the lactose in a suitable mixer.

Mix for about 15 minutes until homogenous.

Continue mixing and add the granulating fluid (sodium/PVP solution).

Mix until a suitable granulation end point is achieved. More IPA can be added to produce a suitable granule.

Dry the granules until an acceptable level of moisture, e.g., less than 1.0% and IPA, e.g., less than 0.5%, is achieved.

Pass the dry granulate through suitable comminution equipment fitted with a suitably sized screen, e.g., 100-500 micron.

Place the granulate in a blender and add the collidal silicon dioxide (glidant), the sodium starch glycolate (disintegrant), and the remaining lactose (diluent) and Avicel (binder diluent).

Mix for about 15 minutes.

Add the magnesium stearate (lubricant) and mix for an additional 5 minutes.

Compress the formulation into oval shaped tablets using a suitable tablet machine.

Alternatively, the metformin is dissolved in IPA (or an alternative solvent) and the PVP is mixed into the dry blend prior to granulation.

### Example 2

### Delayed Release Metformin Formulations

The instant release tablet formulations of Example 1 can be coated with a functional coat. Examples of two types of coatings are given below:

Coating One

| Ingredient | Function | Qty % (w/w) | Batch 1 mg/tab |
|---|---|---|---|
| Eudragit L 100 | Polymer | 6.39 | 6.00 |
| Acetyl Tributyl | Plasticizer | 1.60 | 1.50 |
| Citrate | | | |
| Water* | Solvent | 3.26 | N/A |
| Ethanol* | Solvent | 88.75 | N/A |
| Total | N/A | 100.0 | N/A |

*Removed during processing.

Manufacturing Process

Load the tablets into a suitable coating machine.

Spray the polymer coating on to the tablets.

Once the required amount of polymer coating solution has been applied, dry the tablets in the coating machine.

Coating Two

| Ingredient | Weight (g) |
|---|---|
| Eudragit S 12.5 | 5,000 |
| Dibutyl Sebecate | 125 |
| Talc | 312.5 |
| Purified Water* | 300 |
| Isopropyl Alcohol* | 4262.5 |
| Total | 10,000 |

*Removed during processing.

Manufacturing Process

Add the purified water to the isopropyl alcohol and mix for about 10 minutes.

Add the dibutyl sebecate and stir for about 10 minutes.

Add the talc and continue to mix for about 15 minutes.

Finally, add the Eudragit Sand mix until homogeneous, e.g., about 30 minutes.

Spray directly onto the instant release tablets using fluidized coating equipment and the method described above.

### Example 3

### In Vitro Release Test Results

The delayed release tablets of Example 2 based on coating 1 exhibits the following dissolution profile when tested in a USP type I or II apparatus at 50-100 rpm in 900 ml of medium fluid at 37° C.:
after 2 hours in medium 0.01N HCl <10% of drug is released; and
subsequently after 1 hour in medium pH 6.8 >50% of drug is release.
Subsequently after 2 hour in medium pH 6.8 >75% of drug released

The delayed release tablets of Example 2 based on coating 2 above exhibits a dissolution profile when tested in a USP type I or II apparatus at 50-100 rpm in 900 ml of medium fluid at 37° C.:
after 2 hours in medium 0.01N HCl <10% of drug is released;
subsequently after 1 hour in medium pH 6.8 >10% of drug is released;
   2 hours in medium pH 6.8 >20% of drug is released;
   4 hours in medium pH 6.8 >40% of drug is released; and
   8 hours in medium pH 6.8 >75% of drug is released.

### Example 4

### Modified Release of Metformin Tablet Formulations

Uncoated Modified Release Formulations of Metformin Using Methocel Premium at Various levels. (Wet granulation method).

Matrix Tablet Formulations

The uncoated matrix tablet formulations and processing details are given below:

| | | Formulation | | |
|---|---|---|---|---|
| | | D | E | F |
| Ingredient | Function | mg/tab | mg/tab | mg/tab |
| Metformin | Active | 500.0 | 500.0 | 500.0 |
| Lactose | Diluent | 114.0 | 64.0 | 48.0 |
| Avicel PH101 | Binder Diluent | 124.0 | 74.0 | 58.0 |
| Methocel | Controlled | 200.0 | 300.0 | 400.0 |
| Premium CR** | Release Polymer | | | |
| Colloidal | Glidant | 2.0 | 2.0 | 2.0 |
| Silicon | | | | |
| Dioxide | | | | |
| Magnesium | Lubricant | 10.0 | 10.0 | 10.0 |
| Stearate | | | | |
| PVP | Binder | 50.0 | 50.0 | 50.0 |
| Isopropyl | Solvent | N/A | N/A | N/A |
| Alcohol* | | | | |
| Total (mg) | N/A | 1000 | 1000 | 1068 |

*Removed during processing.
**Methocel grade can be changed or alternatively, a suitable controlled release polymer can be used.

Weigh the ingredients using a suitable balance.

Place metformin, 50% of the Avicel, and 50% of the lactose in a suitable mixer.

Mix for about 15 minutes until homogenous.

Continue mixing and add the granulating fluid (sodium/PVP Solution).

Mix until a suitable granulation end point is achieved. More IPA can be added to produce a suitable granule.

Dry the granules until an acceptable level of moisture, e.g., less than 1.0% and IPA, e.g., less than 0.5%, is achieved.

Pass the dry granulate through suitable comminution equipment fitted with a suitably sized screen, e.g., 100-500 micron.

Place the granulate in a blender and add the collidal silicon dioxide (glidant), and the remaining lactose (diluent) and Avicel (binder diluent).

Mix for about 15 minutes.

Add the magnesium stearate (lubricant) and mix for an additional 5 minutes.

Compress the formulation into oval shaped tablets (target weight about 1000 mg) using a suitable tablet machine.

Alternatively, the metformin is dissolved in IPA (or an alternative solvent) and the PVP is mixed into the dry blend prior to granulation.

### Example 5

### In Vitro Test Results

The above modified-release tablet formulations (D, E, and F) can be coated with a delayed-release functional coating as described in Example 2.

The modified-release tablets of Example 4 based on coating 1 exhibits a dissolution profile when tested in a USP type I or II apparatus at 50-100 rpm in 900 ml of medium fluid at 37° C.:
after 2 hours in medium 0.01N HCl <10% of drug is released;
subsequently after 1 hour in medium pH 6.8 >20% of drug is released;
   2 hours in medium pH 6.8 >30% of drug is released;
   4 hours in medium pH 6.8 >50% of drug is released; and
   8 hours in medium pH 6.8 >75% of drug is released.

The modified release tablets of Example 4 based on coating 2 exhibits a dissolution profile when tested in a USP type I or II apparatus at 50-100 rpm in 900 ml of medium fluid at 37° C.:
after 2 hours in medium 0.01N HCl <10% of drug is released;
subsequently after 1 hour in medium pH 6.8 >10% of drug is released;
   2 hours in medium pH 6.8 >20% of drug is released;
   4 hours in medium pH 6.8 >30% of drug is released;
   6 hours in medium pH 6.8 >40% of drug is released; and
   8 hours in medium pH 6.8 >60% of drug is released.

### Example 6

### Pharmacokinetic Study

A single-dose, five-way crossover study in fifteen healthy volunteers fasting overnight and four hours after dosing is designed to compare and assess the relative bioavailability (the bioavailability obtained by comparing the AUCs when like or unlike dosage forms of the same drug are administered by same or different routes) of four formulations of metformin with a commercial reference product (GLUCOPHAGE®). The formulations are:
(a) GLUCOPHAGE® 500 mg
(b) Delayed Onset 500 mg (A, B or C)
(c) Delayed Onset Modified Release (D)500 mg
(d) Delayed Onset Modified Release (E)500 mg
(e) Delayed Onset Modified Release (F)500 mg

The fifteen healthy volunteers are dosed on one of the 5 study periods in a randomized crossover manner. Venous blood samples are obtained at regular intervals immediately prior to and following each dosing for a period of up to 48 hours. Plasma concentrations of metformin are measured using standard methods. Individual plasma concentration curves are constructed and individual, mean, and relative pharmacokinetic parameters are estimated including Tₘₐₓ (time at the maximum concentration), Cₘₐₓ (maximum observed concentration), and AUC (area under the plasma concentration versus time curve). The following results are obtained:
AUC and Cₘₐₓ of (b)<75% of (a) AUC and Cₘₐₓ
AUC and Cₘₐₓ of (c)<75% of (a) AUC and Cₘₐₓ
AUC and Cₘₐₓ of (d)<60% of (a) AUC and Cₘₐₓ
AUC and Cₘₐₓ of (e)<50% of (a) AUC and Cₘₐₓ

### Example 7

### Clinical Study

A randomized, dose escalation, placebo controlled study is designed to assess the efficacy of the administered formulation in 60 to 120 patients with functional constipation, defined using the Rome II criteria (modified), i.e., at least three weeks in the previous 3 months of two or more of the following symptoms:
i. Straining in >25% of defecations;
ii. Lumpy or hard stools in >25% of defecations;
iii. Sensation of incomplete evacuation in >25% of defecations;
iv. Sensation of anorectal obstruction/blockage in >25% of defecations;
V. Manual maneuvers to facilitate >25% of defecations (e.g. digital evacuation, support of pelvic floor); and/or
vi. <3 evacuations per week.
   In addition, loose stools are not present, and there are insufficient criteria for a diagnosis of IBS. Moreover, these patients have no evidence of medical disorders that can cause constipation. Patients are symptomatic on entry in the randomization phase of the study, i.e., in the 8-14 day run-in period, on at least 8 days, which need not be consecutive, patients have lumpy or hard stools in >25% of defecations.

Patients are randomized to one of three groups:
a) Delayed onset metformin;
b) Delayed onset, modified release metformin; and
c) Placebo.

The primary efficacy endpoint is based on the patient's global impression. Patients receiving metformin answer ‘yes’ to the following question: “do you feel better now after treatment” at least 50% of the time, based on daily diaries, during the dose escalation phase of the study.

Secondary efficacy endpoints include the change from baseline compared to placebo in straining during defecations, stool consistency (Bristol Stool Scale), completeness of evacuation, sensation of anorectal obstruction/blockage, use of manual maneuvers to facilitate defecation, frequency of evacuations, and use of rescue medication, i.e., laxatives.

What is claimed is:

1. A method for treating chronic constipation in a subject in need of such treatment comprising administering to the subject a dosage formulation comprising a therapeutically effective amount of metformin, or a pharmaceutically acceptable salt thereof, and at least one pharmaceutically acceptable ingredient to control the release of the metformin, wherein following administration, the dosage formulation releases the metformin distal to the gastrointestinal sites at which metformin is absorbed.

2. The method according to claim 1, wherein the chronic constipation is a symptom of irritable bowel syndrome.

3. The method according to claim 1, wherein the metformin is administered to the subject orally.

4. The method according to claim 1, wherein the dosage formulation is administered to the subject in a fasting state.

5. The method according to claim 1, wherein the at least one pharmaceutically acceptable ingredient comprises a non-enteric polymer.

6. The method according to claim 1, wherein the at least one pharmaceutically acceptable ingredient comprises an enteric polymer.

7. The method according to claim 1, wherein the dosage formulation is a delayed-release and/or a modified-release formulation.

8. The method according to claim 7, wherein the modified and/or delayed release dissolution profile shows negligible release for at least two hours in a medium with a pH less than or equal to about 5.

9. The method according to claim 7, wherein the modified and/or delayed release dissolution formulation shows negligible release for at least two hours in a medium with a pH less than or equal to about 6.5.

10. The method according to claim 7, wherein the dosage formulation releases the metformin distal to the duodenum of the gastrointestinal tract.

11. The method according to claim 7, wherein the dosage formulation releases the metformin distal to the jejunum of the gastrointestinal tract.

12. The method according to claim 7, wherein the dosage formulation releases the metformin distal to the ileum of the gastrointestinal tract.

13. The method according to claim 1, wherein the dosage formulation releases the metformin after passing through the stomach of the subject.

14. The method according to claim 1, wherein the dosage formulation generates a relative bioavailability of the metformin less than 75% of an administered dose as compared to an immediate release formulation.

15. The method according to claim 14, wherein the dosage formulation generates a relative bioavailability of the metformin less than 50% of an administered dose as compared to an immediate release formulation.

16. The method according to claim 1, wherein the dosage formulation further comprises at least one additional pharmaceutically active compound.

17. The method according to claim 16, wherein the at least one additional pharmaceutically active compound is capable of relieving constipation.

18. The method according to claim 16, wherein the at least one additional pharmaceutically active compound is acarbose.

19. The method according to claim 18, wherein the acarbose is in a form chosen from immediate release and modified release.

20. The method according to claim 1, wherein the dosage formulation is in a tablet form.

21. The method according to claim 1, wherein the dosage formulation provides a daily dose ranging from about 50 mg to about 3 g.

22. The method according to claim 21, wherein the daily dose is chosen from single and divided dosages.

23. The method according to claim 1, wherein the chronic constipation is treated, while minimizing at least one side effect associated with the administration of a conventional formulation of metformin, or a pharmaceutically acceptable salt thereof.

24. A dosage formulation comprising a therapeutically effective amount of metformin, or a pharmaceutically acceptable salt thereof, and at least one pharmaceutically acceptable ingredient to control the release of metformin, wherein the dosage formulation releases metformin distal to the gastrointestinal sites at which metformin is absorbed.

25. The formulation according to claim 24, wherein the at least one pharmaceutically acceptable ingredient comprises a non-enteric polymer.

26. The formulation according to claim 24, wherein the at least one pharmaceutically acceptable ingredient comprises an enteric polymer.

27. The formulation according to claim 24, wherein the dosage formulation is a delayed-release and/or a modified-release formulation.

28. The formulation according to claim 27, wherein the modified and/or delayed release formulation shows negligible release of metformin for at least two hours in a medium with a pH less than or equal to about 6.5.

29. The formulation according to claim 27, wherein the modified and/or delayed release formulation shows negligible release of metformin for at least two hours in a medium with a pH less than or equal to about 5.

30. The formulation according to claim 27, wherein the dosage formulation releases the metformin distal to the duodenum of the gastrointestinal tract.

31. The formulation according to claim 27, wherein the dosage formulation releases the metformin distal to the jejunum of the gastrointestinal tract.

32. The formulation according to claim 27, wherein the dosage formulation releases the metformin distal to the ileum of the gastrointestinal tract.

33. The formulation according to claim 24, wherein the dosage formulation releases the metformin after passing through the stomach of the subject.

34. The formulation according to claim 24, wherein the dosage formulation generates a relative bioavailability of the metformin less than 75% of an administered dose as compared to an immediate release formulation.

35. The formulation according to claim 34, wherein the dosage formulation generates a relative bioavailability of the metformin less than 50% of an administered dose as compared to an immediate release formulation.

36. The formulation according to claim 24, further comprising at least one additional pharmaceutically active compound.

37. The formulation according to claim 36, wherein the at least one additional pharmaceutically active compound is capable of relieving constipation.

38. The formulation according to claim 36, wherein the at least one additional pharmaceutically active compound is acarbose.

39. The formulation according to claim 38, wherein the acarbose is in a form chosen from immediate release and modified release.

40. The formulation according to claim 24, wherein the dosage formulation is in a tablet form.

41. The formulation according to claim 24, wherein the dosage formulation provides a daily dose ranging from about 50 mg to about 3 g.

42. The formulation according to claim 41, wherein the daily dose is chosen from single and divided dosages.

43. A modified-release pharmaceutical tablet comprising a therapeutically effective amount of metformin, or a pharmaceutically acceptable salt thereof, and at least one pharmaceutically acceptable ingredient to control the release of metformin, wherein the modified release tablet exhibits a dissolution profile wherein after about two hours, less than about 10% of the metformin is released in a medium with a pH less than or equal to about 6.5.

44. A modified-release pharmaceutical tablet comprising a therapeutically effective amount of metformin, or a pharmaceutically acceptable salt thereof, and at least one pharmaceutically acceptable ingredient to control the release of metformin, wherein the modified release tablet exhibits a dissolution profile such that after about two hours, less than about 10% of the metformin is released in a medium with a pH less than or equal to about 5.
